(19) United States
(12) Patent Application Publication
(10) Pub. No.: US 2008/0262071 A1
(43) Pub. Date: Oct. 23, 2008

(54) Pindolol for the Treating Premenstrual Syndrome and Premenstrual Dysphoric Disorder

(76) Inventors: Timothy Dinan, Cork (IE); Peter Daly, Dublin (IE)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 11/663,075

(22) PCT Filed: Sep. 19, 2005
(86) PCT No.: PCT/IB05/02769

§ 371 Date: Mar. 18, 2008

Related U.S. Application Data
(60) Provisional application No. 60/610,610, filed on Sep. 17, 2004.
(60) Provisional application No. 60/654,954, filed on Feb. 23, 2005.
(60) Provisional application No. 60/686,718, filed on Jun. 1, 2005.

(51) Int. Cl. A61K 31/404; A61P 15/00; A61P 29/00

(52) U.S. Cl. 514/415

(57) ABSTRACT

In accordance with the present invention, it has been discovered that pindolol provides a highly effective treatment for PMS and/or PMDD. As such, the present invention generally relates to methods and compositions for treating and/or preventing PMS, or symptoms associated therewith including premenstrual tension or premenstrual dysphoria. The methods and compositions of the invention also involves the use of pindolol, a known beta adrenergic blocker, in combination with known contraceptives.

### CROSS-REFERENCES TO RELATED APPLICATIONS

This application claims the benefit of the filing date of U.S. Provisional Application No. 60/610,610, filed Sep. 17, 2004; U.S. Provisional Application No. 60/654,943, filed Feb. 23, 2005; and U.S. Provisional Application No. 60/686,718, filed Jun. 1, 2005, each of which is incorporated herein by reference in its entirety.

### BACKGROUND OF THE INVENTION

The present invention relates to methods and compositions for the treatment of premenstrual syndrome or late luteal phase disorder and related disorders.

Premenstrual syndrome (PMS) or late luteal phase dysphoric disorder (LLPDD) is a common complication of menstruation in otherwise healthy women. Pre-menstrual syndrome is characterized by both affective aspects and physical symptoms. The affective aspects involve in particular affective liability, depressed mood, irritability, lethargy and other features while the physical symptoms often include bloating and breast tenderness.

There are currently only limited treatments for PMS. The related and more serious psychiatric disorder pre-menstrual dysphoric disorder (PMDD) is currently treated by use of selective serotonin reuptake inhibitors (SSRIs), such as fluoxetine and sertraline. These drugs show some efficacy in PMDD with luteal phase administration (Freeman E W, Luteal Phase administration of agents for the treatment of premenstrual dysphoric disorder, CNS Drugs 2004: 18(7): 453-68). However, SSRI's, while effective at the most severe end of this spectrum, do not present a very attractive solution. SSRIs typically have significant side effects which are unacceptable to many patients, including nausea and sexual dysfunction. In addition, the fact that SSRI's are a form of psychiatric treatment serves as a disincentive to many women in accepting treatment, especially those who are less ill than the PMDD patients. As such, it is evident that there is a need for treatment which is effective in treating PMS and PMDD symptoms, is well tolerated, and in addition is preferably not a new indication for an existing psychiatric drug.

More particularly, a systematic review of 15 “high quality” studies of SSRIs in PMS were assessed and the authors concluded that SSRIs may act as a first line treatment for severe PMS. This class of drug treated both the behavioral (irritability, anxiety, depression etc.) as well as the physical symptoms (breast tenderness etc.). They also detected that there was no significant difference in symptom reduction between continuous and intermittent dosing. However, this class of drug is not without side-effects and drop out from treatment due to side-effects was 2.5 times more likely in active treatment groups than in placebo groups. The most common side-effects were nausea, insomnia, dry mouth, dizziness and gastro-intestinal disturbances such as diarrhea, constipation and flatulence. However, their effectiveness does indicate the central and crucial involvement of 5-HT in PMS.

There are a number of other over-the-counter treatments available to treat PMS-associated symptoms such as herbal remedies, calcium supplements and evening primrose oil. Life style changes such as regular intake of carbohydrates, regular exercise, and reduction in alcohol, caffeine and saturated fats have been suggested to improve symptoms. Progesterone and progestogens have been used for the treatment of PMS, but a meta-analysis of 14 double-blind trials did not find a clinically significant difference from placebo treatment (Wyatt et al., Efficacy of progesterone and prostogens in the management of premenstrual syndrome: systemic review, BMJ, 2001: 323:776-83). Gonadorelin analogues have relieved PMS symptoms in several small studies but their use is limited by their hypo-oestrogenic side-effects (hot flushes and osteoporosis) and their high cost.

The involvement of the rennin-angiotensin-aldosterone system has also been suggested as a factor in PMS, and the use of the beta-blocking drug atenolol has been investigated in a PMS study (Rausch, J L, J. Affect Disorder, 1988, September-October: 15(2): 141-7). Atenolol was found to induce significant improvements on the irritability, vigor, elation and friendliness ratings of subjects tested, but significant improvement was not found on several other scales—indicating that atenolol only treated certain symptoms.

In addition, the role of beta 2-adrenergic receptors has been studied in a small sample (n=18) of patients with PMDD, and data was obtained indicating abnormal beta 2-adrenoreceptor regulation (Gurguis et al., Psychiatry Res, 1998, June 2: 79(1): 31-42. Antidepressants have been shown to down-regulate beta adrenergic receptor density and to uncouple the receptor from the G-protein adenyl cyclase system in several studies. Gurguius and co-workers showed higher beta adrenergic receptor density in PMDD patients versus controls.

There is conflicting evidence on whether oral contraception are helpful in the treatment of the symptoms of PMS and PMDD or on some of the symptoms. Some women report that mood deteriorates while using oral contraceptives. However some risk factors have been shown to be associated with OC-related mood deterioration. These include history of depression, dysmenorrhoea, premenstrual depression, higher doses of oestrogen and progestin, use of triphasic preparations as well as family history of OC-related mood changes. Miller and Joffe assessed the risk factors and benefits of using OCs in a questionnaire study of 658 women who had been using OCs for at least three months. No change in mood was found reported by 71.4% of the women; 16.3% reported mood deterioration while 12.3% reported mood improvement. Women who had a history of early onset premenstrual mood disturbance and dysmenorrhoea were more likely to have improved premenstrual mood when taking OCs. (Miller, K E and Joffe H, Am J Obstet Gynecol, December 2003; 189: 1523-30).

Recently a new contraceptive containing ethinyl estradiol and drospirenone has been introduced (brand name “Yasmin”). Drospirenone is a spirinolactone analogue with anti-mineralicorticoid and antiandrogenic properties. This has been studied in PMS. An open label study in 326 women at baseline and cycle 6 found that statistically significant decreases from baseline were found in negative affect and water retention. (Parsey et al., Contraception, 2000, 61:105-111.)

A double blind placebo controlled trial using Yasmin on a sample of 261 women with PMS and PMDD was also conducted. Of 82 women in this PMS group who also fulfilled criteria for PMDD, 42 were in the drospirenone/EE treatment arm. Analysis of factor 1 (mood) showed that active treatment produced a better response but did not reach statistical significance. Statistical significance was reached for three items; increased appetite, food cravings and acne.

Despite these studies, there remains a need for improved methods of treatment of PMS and PMDD.

### BRIEF SUMMARY OF THE INVENTION

In accordance with the present invention, it has been discovered that pindolol, when administered as a racemate (R/S) or in the form of the S-(−) enantiomer, is an effective treatment and/or prevention of premenstrual syndrome (PMS) and symptoms associated therewith, including premenstrual tension and/or premenstrual dysphoria disorder (PMDD).

As such, one aspect of the invention relates to methods for treating and/or preventing premenstrual syndrome (PMS) comprising administering a composition comprising a therapeutically effective amount of at least one pindolol compound of the invention to a subject in need thereof, preferably during the luteal phase of the menstrual cycle.

In accordance with the present invention, it has been discovered that pindolol, when administered to humans in the form of the S-(−) enantiomer, has a reduced effect on lowering blood pressure than racemic pindolol. Such a pharmaceutical composition which is active at the 5HT1a site and has reduced blood pressure lowering effect is useful in the treatment of a number of conditions.

In accordance with the present invention, it has been found that when minus-pindolol is administered to subjects who are healthy from a cardiovascular perspective, that far higher doses can be tolerated than when the racemic mixture is administered. For instance, 10 mg of S(−)-pindolol produced no alteration in blood pressure in a majority of healthy subjects. In marked contrast, the racemic mixture of pindolol at a dose of 10 mg produced definite drops in diastolic blood pressure. This finding was unexpected and is not anticipated in the published literature.

One further aspect of the invention relates to methods for treating and/or preventing premenstrual syndrome (PMS) comprising administering a composition comprising a therapeutically effective amount of S-(−)-pindolol to a subject in need thereof, preferably during the luteal phase of the menstrual cycle.

In yet another aspect of the invention, it has been found that the combination of an oral contraceptive and pindolol provides a highly effective treatment for the symptoms of PMS and PMDD in women who require or desire contraception. Without being bound to any specific mechanism of action the addition of an oral contraceptive and the CNS active drug pindolol acts in additive manner to provide highly effective relief for the physical and psychological symptoms of PMS and PMDD and in particular the most troubling affective and cognitive symptoms. Women taking these drugs showed marked improvement in emotional liability, tension, anxiety and difficulties in concentration as measured by COPE (Calendar of Premenstrual Events). Women taking these drugs also showed improvement in other symptoms such as bloating, breast tenderness, headache and food cravings.

Thus in one embodiment the invention discloses a monophasic, biphasic or triphasic oral contraceptive in combination with, e.g., once a day pindolol or S-pindolol. Optimum benefit may be obtained by use of the pindolol component on a continuous 28 day cycle, i.e. the normal 7 day contraceptive placebo is combined with pindolol or S-pindolol as described herein.

In another aspect of the invention, pharmaceutical compositions are provided which comprise a therapeutically effective amount of at least one pindolol compound of the present invention, together with one or more pharmaceutically acceptable excipients. The pindolol compound can be in a racemic or enantiomerically pure form.

These and other aspects of the invention will become apparent to those skilled in the art.

### DETAILED DESCRIPTION OF THE INVENTION

In accordance with the present invention, it has been discovered that a compound which combines action at both the 5HT1a receptor and at beta-adrenoreceptors will have optimum synergistic effect in relieving a broad range of the symptoms of premenstrual syndrome (PMS). In this regard, as discussed above, the usefulness of SSRI's in PMDD, and other evidence, suggests a serotonergic deficiency in PMS and PMDD.

As such, the present invention generally relates to methods and compositions for treating and/or preventing PMS, or symptoms associated therewith including premenstrual tension or premenstrual dysphoria. The methods and compositions of the invention involve the use of pindolol, a known beta adrenergic blocker.

The involvement of the 5HT1a receptor in PMS has been suggested by the principal co-inventor here (Prof Ted Dinan). The data indicate that 5HT1a receptors are increasingly sensitive in the days prior to menstruation. This hypersensitivity is most likely secondary to fluctuation in sex steroid levels. By way of background Prof Dinan has previously used a similar approach in other inventions in which novel uses for existing drugs are the subject of novel granted patents or patent applications. For instance Prof Dinan has shown previously that pindolol is also useful in the treatment of chronic fatigue syndrome and fibromyalgia (U.S. Pat. No. 6,855,729). U.S. Pat. No. 6,855,729 and the current invention are related in that the drug employed in each case, pindolol or S-pindolol is the same. In addition U.S. Pat. No. 5,403,848 describes the use of pindolol or S-pindolol in Non-Ulcer Dyspepsia.

Moreover it is apparent that PMS and PMDD occur often in the context of a range of psychiatric co-morbidities. PMS and PMDD may occur in association with major depression, cyclothymia, anxiety including generalized anxiety disorder and chronic fatigue syndrome. In addition PMS and PMDD patients may also exhibit physical symptoms such as irritable bowel syndrome, chronic fatigue, fibromyalgia, headache, pain syndromes, other genitor-urinary problems or psychosexual problems and sleep disorders.

Recently there has been much interest in the area of “somatization disorder” which is essentially a form of psychiatric condition of unknown origin which involves a long term history of multiple and varying physical complaints. There are diagnostic criteria for somatization disorder included in DSM-IV and these include; a history of physical complaints over many years and starting before the age of 30; at least four pain symptoms at different sites; two gastro-intestinal symptoms such as nausea or bloating; one sexual or reproductive symptom such as erectile dysfunction or irregular periods and one pseudo-neurological symptom.

It is evident that some PMS/PMDD patients fall into this category and also overlap with patients suffering from IBS and chronic fatigue syndrome/fibromyalgia. The present invention is directed primarily towards PMS and PMDD patients, but also includes patients who are diagnosed as being co-morbid with PMS/PMDD and somatization disorder, IBS, chronic fatigue syndrome and/or fibromyalgia.

A. Compounds of the Invention

The compounds of the invention include pindolol in its racemic (R/S) form, or in its S-(−) enantiomerically pure form. As used herein, “enantiomerically pure” refers to compositions consisting substantially of a single isomer (i.e., substantially free of the opposite isomer), preferably consisting of 90%, 92%, 95%, 98%, 99%, or 100% of a single isomer. In a preferred embodiment, an enantiomerically pure composition comprises at least 95% of a single isomer. In another preferred embodiment, an enantiomerically pure composition comprises at least about 98% of a single isomer. The chemical structure of pindolol is shown below in Formula (I), and the S-isomer is shown in Formula (Ia). As used herein, S-(−) enantiomer pindolol, S-pindolol, minus-pindolol, (−) pindolol, etc., are used interchangeably.

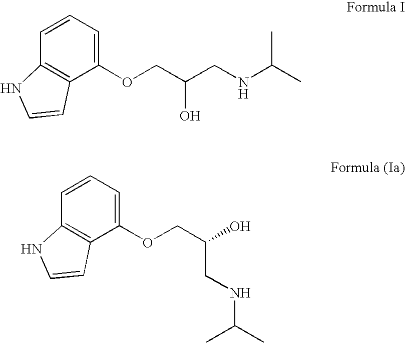

Pindolol is a racemic mixture consisting of both a minus enantiomer and a plus enantiomer. The stereoselectivity of the enantiomers of a range of beta blockers have been studied in relation to both pharmacokinetics and pharmacodynamics. However to date all or almost all studies have reported data from animal sources. Such studies as have been carried out in man involve the determination of pharmacokinetics of the enantiomers following administration of the racemate (such as those reported in the review paper: Mehvar, et al., J Pharm Pharmaceut Sci, 2001, 4(2): 185-200). Such studies show minor differences between (−) and (+) pindolol in areas such pharmacokinetic parameters as AUC, Cmax and t_{1/2} hrs.

Generally the cardiac beta-blocking activity of this class of drugs resides in their S (−) enantiomers. However such studies are based to date on animal data. Thus it has been reported that for pindolol there is a 200:1 selectivity of the (−) enantiomer over the (+) enantiomer in blocking beta-1 and beta-2 receptors in the guinea pig (Jepsson, A B, et al., Acta Pharmacol Toxicol, 1984, 54:285-91).

There are however clearly species differences in the actions of these enantiomers. The comparison of rodent and human beta adrenoceptor function is poorly understood at present. Some hints at such species differences have been apparent in some previous studies also on 5HT1a. For instance, Griebel, et al., J Neuropharmacogy, 2000 Jul. 24: 39(10): 1848-57, found differences in the response of mice and rats in anxiety tests and considered the matter of species differences in 5HT1a receptor function. In rats it would seem that the minus-enantiomer has both 5HT 1a and beta blocking capacity while in man our studies suggest far greater 5HT1a rather than noradrenergic activity.

As mentioned above, pindolol is a known beta adrenergic blocker, and it's preparation and synthesis in racemic or enantiomerically pure form is known in the art. Pindolol has been shown to be effective at both 5HT1a and beta adrenergic sites. Without intending to be limited by theory, it is believed that pindolol exhibits its biological properties primarily through its S-(−) enantiomer. In this regard, it has been demonstrated that in the rat there is a differential tissue distribution of S and R pindolol with the S/R ratio varying from a high of 1.74 for brain to a low of 0.82 in plasma, thus suggesting stereoselective transport of the S (−) pindolol across the blood brain barrier in the rat (Hongmei Yan, et al., European Neuropharmacology, 1999, December: 10(1); 59-62).

By way of background, while pindolol has been tested extensively as an augmentation strategy for antidepressants, the results have been mixed to date. These studies have been reevaluated in the light of recent PET studies that showed that the doses of pindolol used were too low to effect 5HT1a autoreceptor blockade (Rabiner Eugenii A et al., Am J Psychiatry, 2001, 158: 2080-2082). It was shown that in human studies the use of 2.5 mg of pindolol (the dose used in almost all of the previous SSRI studies) did not generate any significant occupancy of 5HT1a receptors while 5.0 mg did produce a modest but significant occupancy of 19%. The dose required for effective SSRI augmentation was stated to be unknown.

Also falling within the scope of the present invention are the in vivo metabolic products of the pindolol compounds described herein. Such products may result for example from the oxidation, reduction, hydrolysis, amidation, esterification and the like of the administered compound, primarily due to enzymatic processes. Accordingly, the invention includes compounds produced by a process comprising contacting a pindolol compound of the invention with a mammalian tissue or a mammal for a period of time sufficient to yield a metabolic product thereof. Such products typically are identified by preparing a radio-labeled (e.g. C¹⁴ or H³) pindolol compound of the invention, administering it in a detectable dose (e.g., greater than about 0.5 mg/kg) to a mammal such as rat, mouse, guinea pig, monkey, or to man, allowing sufficient time for metabolism to occur (typically about 30 seconds to 30 hours), and isolating its conversion products from urine, blood, tumor, or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds of the invention even if they possess no biological activity of their own.

B. Methods of the Invention

In accordance with the present invention, it has been discovered that pindolol, when administered as a racemate (R/S) or in the form of the S-(−) enantiomer, is an effective treatment and/or prevention of PMS and symptoms associated therewith including premenstrual tension or premenstrual dysphoria.

As such, one aspect of the invention relates to methods for treating and/or preventing premenstrual syndrome (PMS) comprising administering a composition comprising a therapeutically effective amount of at least one pindolol compound of the invention to a subject in need thereof, preferably during the luteal phase of the menstrual cycle.

In accordance with other aspects of the present invention, it has been discovered that pindolol, when administered in the form of the S-(−) enantiomer, is effective in the treatment and/or prevention of a number of new indications in which the blood pressure lowering effects of the racemic form may be undesired.

As mentioned above, pindolol is a known beta adrenergic blocker, and is preparation and synthesis in racemic or enantiomerically pure form is known in the art. Pindolol has been shown to be effective at both 5HT1a and beta adrenergic sites. Without intending to be limited by theory, it is believed that pindolol exhibits its biological properties primarily through its S-(−) enantiomer. In this regard, it has been demonstrated that in the rat there is a differential tissue distribution of S and R pindolol with the S/R ratio varying from a high of 1.74 for brain to a low of 0.82 in plasma, thus suggesting stereoselective transport of the S (−) pindolol across the blood brain barrier in the rat (Hongmei Yan et al., European Neuropharmacology, 1999, December: 10(1); 59-62)

Without being bound to any one theory of mechanism of action it is believed from the data presented here that S-(−) pindolol acts in a species specific way in man in relation to its antagonism of the beta-1 and beta-2 receptors. In any event, in accordance with the invention, it has been unexpectedly found that effects on blood pressure are less using the S-(−) enantiomer of pindolol as compared to the racemate.

As such, one aspect of the invention relates to methods for treating and/or preventing premenstrual syndrome (PMS) comprising administering a composition comprising a therapeutically effective amount of S-(−)-pindolol to a subject in need thereof, preferably during the luteal phase of the menstrual cycle.

This invention provides for a once a day form of pindolol in combination with a once a day oral contraceptive for the treatment of symptoms of premenstrual syndrome in women who require or are desirous of contraception.

As such, one aspect of the invention relates to methods for treating and/or preventing PMS and/or PMDD in a subject desirous of contraception, comprising administering a composition comprising an amount of at least one pindolol compound of the invention effective to treat and/or prevent PMS or PMDD to a subject desirous of contraception in combination with a contraceptive. The pindolol compound may be administered throughout the menstrual cycle, or may be administered during the luteal phase of the menstrual cycle. Further, the pindolol compound and the contraceptive may be administered as a single, fixed dose composition, or may be administered sequentially as separate compositions.

Thus in one aspect, the invention provides administration of a monophasic, biphasic or triphasic oral contraceptive in combination with either racemic pindolol or S-pindolol. In one embodiment, the pindolol component may be administered on a continuous 28 day cycle, wherein the normal 7 day contraceptive placebo is combined with the racemic pindolol or S-pindolol as described herein.

In another aspect, the invention provides a combination therapy which may be utilized by subjects with an increased risk of cardiovascular events and who are desirous of contraception, such as oral contraception, transdermal “patch” contraception, or the like. The slight decrease in blood pressure provided by the methods as described herein provide a useful reduction in risk factors in this population.

In one embodiment, the invention employs once a day administration of pindolol in a fixed dose combination with a once a day oral contraceptive for the treatment of symptoms of PMS or PMDD in women who are desirous of contraception. In a further embodiment the invention comprises a once a day form of pindolol or S-pindolol which has anti-minericorticoid properties.

In another embodiment, the invention comprises a once a day administration of racemic pindolol or S-pindolol in combination with an oral contraceptive comprising oestrogen or an analogue thereof and a progestin. The racemic pindolol or S-pindolol may be administered throughout the entire cycle. In another embodiment, the invention comprises a once a day form of racemic pindolol or S-pindolol in combination with a progestin only contraceptive.

In an alternative embodiment, the pindolol may be administered in combination with the contraceptive as a transdermal patch or implant.

In one embodiment the invention comprises a once a day form of racemic pindolol or S-pindolol in combination with a combined oral contraceptive containing oestrogen or a analogue and a progestin. The pindolol or S-pindolol is administered throughout the entire cycle.

In another embodiment the invention comprises a once a day form of racemic pindolol or S-pindolol in combination with a progestin only contraceptive.

In a further embodiment the invention comprises a once a day form of pindolol or S-pindolol which has antiminericorticoid properties.

In yet another embodiment of the invention, the combination drug is useful in the treatment of post-menopausal symptoms. The combination of drospirenone plus ethinyl estradiol plus either pindolol or S-pindolol provides treatment for hot flushes associated with withdrawal of sex steroids and the small drop in blood pressure resulting from pindolol provides for a lowering of cardiovascular risk in this population. The CNS effects of pindolol or S-pindolol additionally provide for treatment of anxiety in this group.

In yet another aspect, the invention provides a combination therapy which may be effectively utilized by women in the perimenopausal, menopausal, and post-menopausal periods. It is known that PMS is a predictor of troublesome perimenopausal, menopausal, and post-menopausal symptoms including hot flashes and depression.

As such, in yet another embodiment of the invention, the combination therapy is useful in the treatment of perimenopausal, menopausal, and post-menopausal symptoms. By way of example, drospirenone and ethinyl estradiol in combination with either racemic pindolol or S-pindolol provides treatment for hot flushes associated with withdrawal of sex steroids. Further, the small drop in blood pressure resulting from pindolol provides for a lowering of cardiovascular risk in this population. The CNS effects of racemic pindolol or S-pindolol additionally provide for treatment of anxiety in this group.

Another aspect of the invention relates to the use of the drugs of the invention in the treatment of patients who present with PMS or PMDD and other psychiatric or somatic co-morbidities. Based on previous inventions the drugs of the invention are useful in the treatment of patients who are suffering from anxiety, chronic fatigue syndrome, fibromyalgia or other pain syndromes in association with PMS or PMDD. In addition the drugs of the invention may be employed in the treatment of patients who have a dual diagnosis of PMS or PMDD in association with somatization disorder.

According to the methods of the invention, the compound(s) may be administered to the subject via any drug delivery route known in the art. Specific exemplary administration routes include peripheral and central routes such as oral, ocular, rectal, buccal, topical, nasal, ophthalmic, subcutaneous, intramuscular, intravenous (bolus and infusion), intracerebral, transdermal, and pulmonary. In a preferred embodiment, the composition is administered as a transdermal patch using methods known in the art.

The term “therapeutically effective amount” and “prophylactically effective amount”, as used herein, refers to an amount of a pharmaceutical agent to treat, ameliorate, or prevent the identified disease or condition, or to exhibit a detectable therapeutic or inhibitory effect, respectively. The effect can be detected by any means known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

More particularly, preferred therapeutically effective amounts of the compound(s) of the present invention include an initial target plasma concentration ranging from approximately 5 μg/mL to approximately 100 μg/mL, preferably from approximately 10 μg/mL to approximately 50 μg/mL, more preferably from approximately 10 μg/mL to approximately 25 μg/mL. To achieve such plasma concentrations, the compounds of the invention may be administered at doses that vary from 0.1 μg to 100,000 mg, depending upon the route of administration. Guidance as to particular dosages and methods of delivery is provided in the literature and is generally available to practitioners in the art. In general the dose will be in the range of about 0.1 mg/day to about 10 g/day, or about 0.5 mg to about 3 g/day, or about 1 mg to about 3 g/day, or about 1 mg to about 50 mg/day, or about 2 mg/day to about 25 mg/day, in single, divided, or continuous doses for a patient weighing between about 40 to about 100 kg (which dose may be adjusted for patients above or below this weight range, particularly children under 40 kg).

The exact dosage will be determined by the practitioner, in light of factors related to the subject that requires treatment and the form of pindolol compound used (e.g., racemic or S-(−) enantiomeric form). Dosage and administration are adjusted to provide sufficient levels of the active agent(s) or to maintain the desired effect. Dosage amounts may be adjusted depending on whether the pindolol compound is administered as a racemic mixture or as an S-(−) enantiomerically pure form. As discussed above, it is believed that the S-(−) enantiomer of pindolol is primarily responsible for the biological activity of pindolol. Thus, if enantiomerically pure compositions are used, the dosage will generally be lower than if racemic mixtures are used. Other factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

C. Pharmaceutical Compositions of the Invention

In yet another aspect of the invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise a therapeutically effective amount of at least one pindolol compound of the present invention, together with one or more pharmaceutically acceptable excipients. For instance, when the pharmaceutical composition is formulated as an oral tablet, the composition preferably comprises from about 0.1 mg to about 25 mg of the pindolol compound, more preferably from about 5 mg to about 25 mg. As discussed above, the exact amount of the pindolol compound may vary depending on the form of pindolol compound used (e.g., racemic or enantiomeric).

Optionally, the pharmaceutical compositions of the invention may comprise a combination of pindolol compounds of the present invention, or may include a second therapeutic agent useful in the treatment of PMS or associated symptoms. Therapeutic amounts of second agents are generally known in the art or may be determined by the skilled clinician.

In another embodiment, the pharmaceutical compositions of the invention may comprise contraceptives, as is known in the art. For instance, the compositions may comprise monophasic, biphasic or triphasic contraceptives, such as oestrogen or an analogue thereof and a progestin, or may comprise a progestin only. Alternatively, the compositions may comprise therapeutic agents useful in the treatment of perimenopausal, menopausal, or post-menopausal symptoms, such as drospirenone and ethinyl estradio.

Formulations of the present invention, e.g., for parenteral or oral administration, are most typically solids, liquid solutions, emulsions or suspensions, while inhalable formulations for pulmonary administration are generally liquids or powders, with powder formulations being generally preferred. Alternative pharmaceutical compositions of the invention may be formulated as syrups, creams, ointments, tablets, and the like.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

D. Combination Therapy

It is also possible to combine any pindolol compound of the present invention with one or more other active agents useful in the treatment of PMS, or its associated symptoms, in a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

The skilled artisan will recognize that a variety of active ingredients may be administered in combination with the compounds of the present invention that may act to augment or synergistically enhance the activity of the pindolol compounds of the invention.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

### Example 1

A single blind pilot study to evaluate the safety, efficacy and tolerability of pindolol in PMS was initiated at one study centre. The trial objectives were to: (1) To determine the efficacy of pindolol in ameliorating PMS (Premenstrual syndrome) symptoms as measured by the Calendar of Premenstrual Experiences which is a diary of 22 physcial and “behavioral” symptoms of PMS; (2) To determine the tolerability of pindolol in PMS; (3) To determine the safety of pindolol in PMS; and (4) To establish a dose range of pindolol which can be used in a later double blind trial.

Twenty subjects were recruited who met the criteria for inclusion in the study. After screening subjects underwent a one month assessment period with prospective diaries (COPE, Calendar of Premenstrual Events). By calculating the subjects COPE scores in the follicular and luteal phases, a diagnosis of late luteal phase dysphoric disorder could be made. All subjects then entered the single-blind placebo treatment period and the subjects' outcome at the end of this cycle acted as their baseline assessment. Subjects were then treated with pindolol for the next three cycles and assessed using the COPE. During this three month period, subjects may have a sequential increase in dose at monthly intervals from 15 to 25 mg/day to optimize response. Provision was made for dose decreases in case of tolerability concerns. Subjects were aware that they would receive three months of active and one month of placebo but were unaware of when the placebo was administered. The primary outcome measure was the mean change from the end of the placebo period in luteal phase total COPE score after three menstrual cycles of active treatment (pindolol). Secondary efficacy measures included; mean change from the end of the placebo period in luteal phase total COPE score; mean change from end of the placebo period in luteal phase physical COPE symptoms; mean change from the end of the luteal phase behavioral symptoms; mean change from end of the placebo period in follicular phase total COPE score; CGI improvement score at each of the three menstrual cycles.

There was a decrease in the reduction of total COPE score in all patients treated with active drug. Significant decreases in total COPE score were seen in patients who were on doses of 15 mg and above. Particularly significant decreases were observed in the behavioral COPE scores and on improvement in CGI. The drug was very well tolerated and no withdrawals for reasons of unacceptable reductions in blood pressure were noted.

### Example 2

Ten healthy male volunteers were tested on two separate occasions one week apart. On both occasions their blood pressure was measured at baseline. On a randomized basis they were given on one occasion pindolol 10 mg orally and on another occasion (−) pindolol 10 mg orally. Within two hours of pindolol administration a definite drop in blood pressure was detected. In all subjects (−) pindolol 10 mg produced no alteration in blood pressure.

### Example 3

Patient was a 26 year old woman with a long history of premenstrual dysphoric disorder. She was treated for 3 months as follows: Month 1: triphasic oral contraceptive; Month 2: (−) Pindolol 5 mg three times daily throughout luteal phase; Month 3: Combination therapy of triphasic pill and (−) Pindolol in the luteal phase.

Throughout each cycle, a daily diary was used to document symptoms. In month 1, the oral contraceptive reduced physical symptoms such as breast tenderness. In month 2, (−) Pindolol helped improve mood and reduce anxiety. The greatest improvement took place in month 3 with a reduction in both physical and psychological symptoms.

Having described several embodiments, it will be recognized by those of skill in the art that various modifications, alternative constructions, and equivalents may be used without departing from the spirit of the invention. Accordingly, the above description should not be taken as limiting the scope of the invention, which is defined in the following claims.

1.-21. (canceled)

22. A pharmaceutical composition comprising a therapeutically or prophylactically effective amount of enantiomerically pure S-(−) pindolol and at least one pharmaceutically acceptable excipient.

23. The pharmaceutical composition of claim 22, wherein said composition comprises an amount of enantiomerically pure S-(−) pindolol, such that at least about 1 mg to about 50 mg is delivered to a subject when administered in single or divided doses.

24. A method of treating and/or preventing pre-menstrual syndrome (PMS) and/or symptoms associated therewith, said method comprising administering a therapeutically or prophylactically effective amount of a pindolol compound selected from racemic pindolol or enantiomerically pure S-(−) pindolol to a subject in need thereof.

25. A method according to claim 24, wherein said symptoms associated with PMS are selected from the group consisting of late luteal phase dysphoric disorder (LLPDD) and premenstrual dysphoric disorder (PMDD).

26. A method according to claim 24, wherein said treatment and/or prevention occurs during the luteal phase of the menstrual cycle.

27. A method according to claim 24, wherein said pindolol compound is enantiomerically pure S-(−) pindolol.

28. A method according to claim 24, wherein said pindolol compound is racemic pindolol.

29. A method according to claim 24, wherein about 1 mg to about 50 mg of said pindolol compound is administered to said subject.

30. A method according claim 24, wherein about 2 mg to about 50 mg of racemic pindolol is administered to said subject.

31. A method according to claim 24, wherein about 1 mg to about 25 mg of enantiomerically pure S-(−) pindolol is administered to said subject.

32. A method according to claim 24, wherein said subject is diagnosed with one or more of chronic fatigue syndrome, fibromyalgia, other chronic pain syndromes, and/or somatization disorders.

33. A method for the treatment and/or prevention of pre-menstrual syndrome (PMS) and/or symptoms associated therewith, said method comprising administering a therapeutically or prophylactically effective amount of a pindolol compound selected from racemic pindolol or enantiomerically pure S-(−) pindolol, in combination with a contraceptive, to a subject in need thereof.

34. A method according to claim 33, wherein said subject has an increased risk of cardiovascular events, and wherein said pindolol compound is administered in an amount effective to decrease the blood pressure of said subject.

35. A method according to claim 33, wherein said contraceptive is a monophasic, biphasic or triphasic oral contraceptive.

36. A method according to claim 33, wherein about 1 mg to about 50 mg of said pindolol compound is administered to said subject.

37. A method according to claim 33, wherein about 1 mg to about 25 mg of enantiomerically pure S-(−) pindolol is administered to said subject.

38. A method according to claim 33, wherein said pindolol compound and said contraceptive are formulated as a single fixed dose composition.

39. A method according to claim 33, wherein said contraceptive is administered separately from said pindolol compound.

40. A method according to claim 33, wherein said subject is diagnosed with one or more of chronic fatigue syndrome, fibromyalgia, other chronic pain syndromes, and/or somatization disorders.

41. A method for the treatment and/or prevention of pre-menstrual syndrome or pre-menstrual dysphoric disorder, and symptoms associated therewith, said method comprising administering a therapeutically or prophylactically effective amount of a pindolol compound selected from racemic pindolol or enantiomerically pure S-(−) pindolol, in combination with a contraceptive, to a subject in need thereof.

42. A method according to claim 41, wherein said subject has an increased risk of cardiovascular events, and wherein said pindolol compound is administered in an amount effective to decrease the blood pressure of said subject.

43. A method according to claim 41, wherein said contraceptive is a monophasic, biphasic or triphasic oral contraceptive.

44. A method according to claim 41, wherein about 1 mg to about 50 mg of said pindolol compound is administered to said subject.

45. A method according to claim 41, wherein about 1 mg to about 25 mgs of enantiomerically pure S-(−) pindolol is administered to said subject.

46. A method according to claim 41, wherein said pindolol compound and said contraceptive are administered as a single fixed dose composition.

47. A method according to claim 41, wherein said contraceptive is administered separately from said pindolol compound.

48. A method according to claim 41, wherein said subject is diagnosed with one or more of chronic fatigue syndrome, fibromyalgia, other chronic pain syndromes, and/or somatization disorders.

49. A method for the treatment and/or prevention of perimenopausal, menopausal, and/or post-menopausal symptoms, said method comprising administering a therapeutically or prophylactically effective amount of a pindolol compound selected from racemic pindolol or enantiomerically pure S-(−) pindolol, in combination with a contraceptive, to a subject in need thereof.

50. A method according to claim 49, wherein said subject has an increased risk of cardiovascular events, and wherein said pindolol compound is administered in an amount effective to decrease the blood pressure of said subject.

51. A method according to claim 49, wherein said contraceptive is a monophasic, biphasic or triphasic oral contraceptive.

52. A method according to claim 49, wherein about 1 mg to about 50 mg of said pindolol compound is administered to said subject.

53. A method according to claim 49, wherein about 1 mg to about 25 mgs of enantiomerically pure S-(−) pindolol is administered to said subject.

54. A method according to claim 49, wherein said pindolol compound and said contraceptive are administered as a single fixed dose composition.

55. A method according to claim 49, wherein said contraceptive is administered separately from said pindolol compound.

56. A method according to claim 49, wherein said subject is diagnosed with one or more of chronic fatigue syndrome, fibromyalgia, other chronic pain syndromes, and/or somatization disorders.

57. A method for the treatment and/or prevention of a condition or disorder involving super sensitivity of the 5HT1a receptor, said method comprising administering a therapeutically or prophylactically effective amount of an enantiomerically pure S-(−) pindolol to a subject in need thereof in an amount such that said enantiomer may be administered safely in higher doses than racemic pindolol.

58. A method according to claim 57, wherein the condition or disorder is selected from the group consisting of premenstrual syndrome, fibromyalgia, and chronic fatigue syndrome.

59. A method according to claim 57, wherein between about 1 mg to about 50 mg of enantiomerically pure S-(−) pindolol is administered to said subject.
